(19) United States
(12) Patent Application Publication
(10) Pub. No.: US 2026/0227378 A1
(43) Pub. Date: Aug. 6, 2026

(54) A HEALTH MONITORING OR DIAGNOSTIC SYSTEM COMPRISING A READER UNIT

(71) Applicant: TACHMED HOLDINGS LIMITED, Guildford (GB)

(72) Inventors: Paul CHRISTIE, Guildford (GB); Glen TYRRELL, Guildford (GB)

(21) Appl. No.: 19/115,640

(22) PCT Filed: Sep. 25, 2023
(86) PCT No.: PCT/GB2023/052479

§ 371 Date: Mar. 26, 2025

(30) Foreign Application Priority Data
Sep. 26, 2022 (GB) 2214040.4
Jun. 20, 2023 (GB) 2309274.5

(51) Int. Cl. G01N 33/487; G16H 10/40; G16H 40/63

(52) U.S. Cl. CPC G01N 33/48785; G01N 33/48707; G16H 10/40; G16H 40/63

(57) ABSTRACT

A diagnostics system comprising: a reader unit configured to connect to, and read a signal from, a sensor unit configured to sense a biomarker within a biological sample, the reader unit comprising a control unit configured to control the reader unit; a user computer device configured to be operated by a user of the diagnostic system and configured to exchange data with and provide instructions to the control unit for controlling the reader unit, wherein the control unit is configured to communicate information about an identity of the sensor unit to the user computer device and the user computer device is configured to provide instructions to the control unit for controlling the reader unit based on the identity of the sensor unit.

### TECHNICAL FIELD

The present disclosure relates to a health monitoring or diagnostics system.

### BACKGROUND ART

At present, medical diagnostic testing, for monitoring a state of health or diagnosing disease, often requires a specialist to take a sample from a patient, which is sent to another specialist to test using specialist lab-based equipment, then the results are sent to another specialist to interpret. This process is slow, has a high administrative burden and is susceptible to human error in the process.

The present disclosure aims to at least partially solve the above problems.

### SUMMARY OF THE INVENTION

According to an aspect of the disclosure there is provided a health monitoring or diagnostics system comprising: a reader unit configured to connect to, and read a signal from, a sensor unit configured to sense a biomarker within a biological sample, the reader unit comprising a control unit configured to control the reader unit; a user computer device configured to be operated by a user of the diagnostic system and configured to exchange data with and provide instructions to the control unit for controlling the reader unit,

wherein the control unit is configured to communicate information about an identity of the sensor unit to the user computer device and the user computer device is configured to provide instructions to the control unit for controlling the reader unit based on the identity of the sensor unit.

Optionally, the control unit is configured to control the reader unit to provide a voltage to the sensor unit, the instructions to the control unit for controlling the reader unit include a level of the voltage, and the level of the voltage is determined based on the information about the identity of the sensor unit.

Optionally, the control unit is configured to control the reader unit to perform signal processing on a signal received from the sensor unit, the instructions to the control unit for controlling the reader unit include a type of signal processing, and the type of signal processing is based on the information about the identity of the sensor unit. Optionally, the signal processing includes comparison of a signal from the sensor unit with baseline sensor data, the instructions to the control unit for controlling the reader unit include the baseline sensor data, and the baseline sensor data is based on the information about the identity of the sensor unit.

Optionally, the user computer device is configured to determine the type of sensor unit, based on the information about the identity of the sensor unit, and provide instructions to the control unit based on the type of the sensor unit.

Optionally, the user computer device is configured to determine whether the user is authorised to use the sensor unit, based on the information about the identity of the sensor unit, and provide instructions to the control unit based on whether the user is authorised to use the sensor unit.

Optionally, the user computer device is configured to determine whether the sensor unit is valid, based on the information about the identity of the sensor unit, and provide instructions to the control unit based on whether the sensor unit is valid.

Optionally, the control unit is configured to communicate information about an identity of the reader unit to the user computer device and the user computer device is configured to provide instructions to the control unit for controlling the reader unit based on the identity of the reader unit. Optionally, the user computer device is configured to determine whether the user is authorised to use the reader unit, based on the information about the identity of the sensor unit, and provide instructions to the control unit based on whether the user is authorised to use the sensor unit. Optionally, the user computer device is configured to determine whether the reader unit is valid, based on the information about the identity of the sensor unit, and provide instructions to the control unit based on whether the sensor unit is valid.

Optionally, the instructions provided by the user computer user device to the control unit are based on data from the host computer device. Optionally, the host computer device is configured to provide to the user computer device data relating to one or more of: baseline sensor data, validity of the sensor unit, authorisation for the user to use the sensor unit, validity of the reader unit, authorisation for the user to use the sensor unit.

Optionally, the reader unit and the sensor unit comprise respective interfaces configured to provide a connection between the reader unit and the sensor unit, the respective interfaces being configured to detachably connect such that the sensor unit can be interchanged with different sensor unit.

Optionally, the reader unit is operable in a plurality of different modes, each different mode corresponding to a different type of sensor unit.

Optionally, the user computer device is provided separately from the reader unit and is connected wirelessly thereto.

Optionally, the system is configured to determine, based on the signal from the sensor unit, whether the biological sample is indicative of a particular state of health or disease. Optionally, the control unit, the user computer device, or the host computer device is configured to determine, based on the signal from the sensor unit, whether the biological sample is indicative of a particular state of health or disease. Optionally, the determination is provided to the user computer device from the control unit or the host computer device.

According to another aspect of the disclosure there is provided a health monitoring or diagnostics method comprising: connecting a sensor unit configured to sense a biomarker within a biological sample to a reader unit comprising a control unit configured to control the reader unit; the control unit communicating information about an identity of the sensor unit to a user computer device operated by a user of the diagnostic system, and the user computer device providing instructions to the control unit for controlling the reader unit based on the identity of the sensor unit; and the reader unit reading a signal from the sensor unit.

### BRIEF DESCRIPTION OF THE DRAWINGS

Further features of the disclosure will be described below, by way of non-limiting examples and with reference to the accompanying drawings, in which:

FIG. 1 schematically shows an example diagnostics system according to the disclosure;

FIG. 2 schematically shows the example diagnostics system of FIG. 1 in further detail;

FIG. 3 schematically shows the reader unit and the sensor unit in further detail;

FIG. 4 schematically shows the example diagnostics system of FIG. 1 in further detail;

FIG. 5 schematically shows an example sensor unit;

FIG. 6 shows the exchange of data within the diagnostics system;

FIG. 7 shows an example reader unit with hub unit and adapter unit disconnected;

FIG. 8 shows the example reader unit of FIG. 7 with hub unit and adapter unit connected;

FIG. 9 shows an example diagnostics system comprising the reader unit of FIG. 7 and a sensor unit;

FIG. 10 shows an exploded view of example sensor unit shown in FIG. 9;

FIG. 11 shows another view of the example sensor unit shown in FIG. 9;

FIG. 12 shows part of the example sensor unit shown in FIG. 9 with partial transparency to show internal components; and

FIG. 13 shows a cross-sectional view of part of the sensor unit shown in FIG. 9.

### DETAILED DESCRIPTION

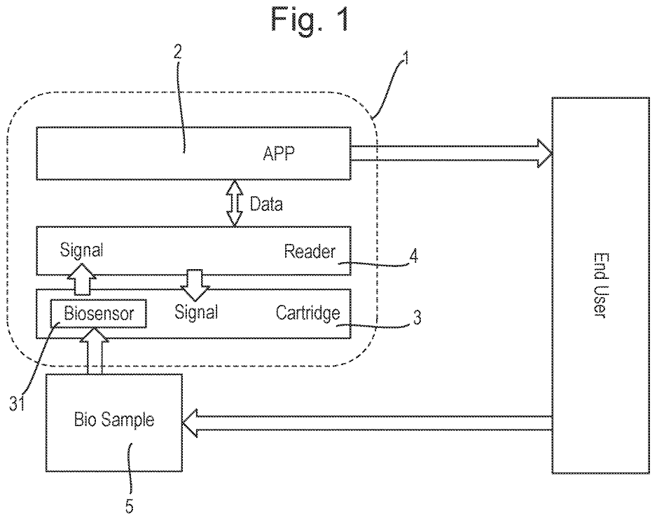

FIG. 1 shows an example health monitoring or diagnostics system 1 (hereinafter diagnostics system) according to the disclosure. As shown, the system may comprise a reader unit 4 (labelled Reader), a sensor unit 3 (labelled Cartridge) and a user computer device 2 (labelled APP).

The reader unit may be configured to connect to, and read a signal from, the sensor unit. The reader unit may comprise a control unit configured to control the reader unit. The sensor unit may be configured to sense a biomarker within a biological sample 5 (labelled Bio Sample). The sensor unit may comprise a biosensor 31 (labelled Biosensor, also referred to as a sensor) configured to sense the biomarker within the biological sample.

Although not shown in FIG. 1, the reader unit and the sensor unit may comprise respective interfaces configured to provide a connection between the reader unit and the sensor unit. The respective interfaces may be configured to detachably connect such that the sensor unit can be interchanged with different sensor unit. The respective interfaces may comprise a mechanical connection, power connection, and/or data connection between the reader unit and the sensor unit. The power connection may be an electrical connection. The data connection may be an electrical connection or an optical connection, for example.

The sensor unit may be configured to be inserted into the reader unit to connect the reader unit and the sensor unit, for example. The reader unit may comprise a housing in which the components forming the reader unit are arranged, including the control unit and the interface. The housing may comprise an opening, or orifice into which the sensor unit is configured to be inserted.

As shown in FIG. 1, the reader unit may provide a signal to the senor unit for sensing the biomarker within the biological sample. As shown, the sensor unit may provide a signal to the reader relating to the sensed biomarker within the biological sample. The signals may be electrical signals, for example.

As shown in FIG. 1, the user computer device may be configured to be operated by a user of the diagnostics system. The user computer device may be configured to exchange data with the reader unit. The user computer device may be configured to provide instructions to the control unit of the reader unit for controlling the reader unit.

The user computer device may be separate from the reader unit and the sensor unit. For example, the user computer device may be a remote device. The user computer device may be a handheld device. For example, the user computer device may be a smartphone or a tablet computer.

The user computer device may be configured to run specific software (e.g. a software application, or app, including an API) that enables the exchange of data with the reader unit. A user may have a user identification (ID) associated with the software. The software may thus be configured to the user.

As shown in FIG. 1, the user (labelled End User) may provide the biological sample and the same user may interact with and/or control the diagnostics system through the user computer device. In other examples different users may provide the biological sample and interact with and/or control the diagnostics system through the user computer device.

The biological sample may be from a human. In other examples, the biological sample may be from an animal or plant. If from an animal or plant, the provider of the biological sample may not be the user of the system.

Although not shown in FIG. 1, the system may comprise a host computer device, separate from the user computer device. The host computer device may be configured to exchange data with the user computer device. For example, the instructions provided by the user computer user device to the control unit may be based on data from the host computer device. The host computer device may be a remote computer, or plurality of remote computers (e.g. servers) in the same location or different locations. The remote computer may be cloud based computer providing cloud computing.

The diagnostics system may be configured to enable and/or prevent a user from using the system based on the data exchange between the user computer device and the reader unit. for example, the diagnostics system may be configured to enable and/or prevent the sensor unit sensing a biomarker within a biological sample, based on the data exchange between the user computer device and the reader unit.

The control unit may be configured to communicate information about an identity of the sensor unit to the user computer device. The user computer device may be configured to provide instructions to the control unit for controlling the reader unit based on the identity of the sensor unit. The information about the identity of the sensor unit may be an identification number (e.g. serial number) associated with the sensor unit. The information about the identity of the sensor may identify a specific single sensor unit and/or may identify a type of the sensor unit. Different information may identify the sensor unit and the type of the sensor unit.

The type of the sensor unit may refer to its operation and/or the type of biomarker it is configured to sense and/or the type of biological sample it is configured to sense the biomarker in. For example, two different types of sensor unit may be configured to sense the same biomarker from the same type of biological sample, but operate differently. Another two different types of sensor unit may be configured to sense the same biomarker from different types of biological sample. Another two different types of sensor unit may be configured to sense the different biomarkers from the same type of biological sample. Another two different types of sensor unit may be configured to sense different biomarkers from different types of biological sample.

The information about an identity of the sensor unit may be communicated to the reader unit by a data connection between the sensor unit and the reader unit. This may be an electrical data connection, for example. The information about an identity of the sensor unit may be stored in a memory of the data unit. Alternatively, information about an identity of the sensor unit may be communicated to the reader unit by radio-frequency identification (RFID). For example, the sensor unit may comprise an RFID tag. This may be passive RFID tag that does not require its own power supply. Alternatively, information about the identity of the sensor unit may be communicated to the reader unit by optical means, e.g. by computer vision or a barcode reader. Corresponding components may be provided to the sensor device and the reader device to enable such communication.

The user computer device may be configured to determine whether the user is authorised to use the sensor unit, based on the information about the identity of the sensor unit. The user computer device may be configured to provide instructions to the control unit based on whether the user is authorised to use the sensor unit. For example, the user may be authorised if the user ID associated with software on the user computer device corresponds with a valid ID stored in a database. The database may be stored on the user computer device and/or on a host computer device in communication with the user computer device. Alternatively, the user may be authorised to use the sensor unit if the user ID associated with software on the user computer device is associated with an ID of the sensor unit in a database. The database may be stored on the user computer device and/or on a host computer device in communication with the user computer device. In some examples, the user computer device may enable the user ID to be associated with the ID of the sensor unit, if not already associated, to provide authorisation. In other words, the sensor unit may be registered to the user.

The user computer device may be configured to determine whether the sensor unit is valid, based on the information about the identity of the sensor unit. The user computer device may be configured to provide instructions to the control unit based on whether the sensor unit is valid. For example, the sensor unit may be valid if an ID of the sensor unit corresponds with a valid ID stored in a database. The database may be stored on the user computer device and/or on a host computer device in communication with the user computer device.

The control unit may be configured to communicate information about an identity of the reader unit to the user computer device. The user computer device may be configured to provide instructions to the control unit for controlling the reader unit based on the identity of the reader unit. The information about the identity of the reader unit may be an identification number (e.g. serial number) associated with the sensor unit. The information about the identity of the sensor may identify a specific reader unit and/or may identify a type of the reader unit. Different information may identify the reader unit and the type of the reader unit. The type of the reader unit may refer to its operation and/or the type of sensor unit it is configured to be used with.

The user computer device may be configured to determine whether the user is authorised to use the reader unit, based on the information about the identity of the reader unit. The user computer device may be configured to provide instructions to the control unit based on whether the user is authorised to use the reader unit. For example, the user may be authorised if the user ID associated with software on the user computer device corresponds with a valid ID stored in a database. The database may be stored on the user computer device and/or on a host computer device in communication with the user computer device. Alternatively, the user may be authorised to use the reader unit if the user ID associated with software on the user computer device is associated with an ID of the reader unit in a database. The database may be stored on the user computer device and/or on a host computer device in communication with the user computer device. In some examples, the user computer device may enable the user ID to be associated with the ID of the reader unit, if not already associated, to provide authorisation. In other words, the reader unit may be registered to the user.

The user computer device may be configured to determine whether the reader unit is valid, based on the information about the identity of the reader unit. The user computer device may be configured to provide instructions to the control unit based on whether the reader unit is valid. For example, the reader unit may be valid if an ID of the reader unit corresponds with a valid ID stored in a database. The database may be stored on the user computer device and/or on a host computer device in communication with the user computer device.

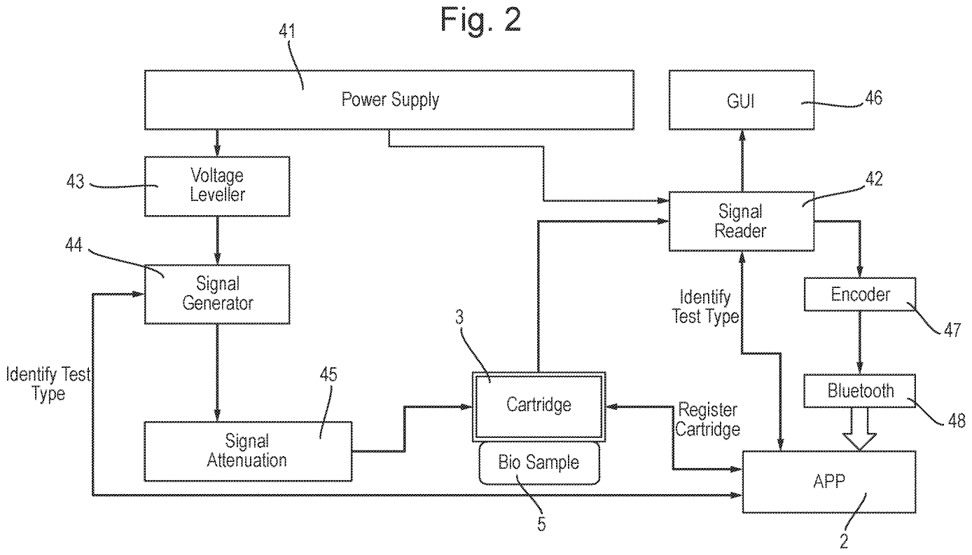

FIG. 2 shows the example diagnostics system of FIG. 1 in further detail. As shown the system may comprise a power supply 41 (labelled Power Supply). The power supply may be provided to the reader unit. As shown, the power supply may power a signal reader 32 (labelled Signal Reader) of the reader unit configured to read a signal from the sensor unit 3 (labelled Cartridge). As shown, the power supply may also provide power to the sensor unit. As shown, the power supply may provide a signal to the sensor unit. As shown this may be provided by signal generating electrical components, such as a voltage leveller 43 (labelled Voltage Leveller), a signal generator 44 (labelled Signal Generator) and a signal attenuator 45 (labelled Signal Attenuator). Although not shown, the signal may be controlled by the control unit of the reader unit.

As shown in FIG. 2, the signal reader may provide data to a graphical user interface 46 (labelled GUI) of the reader unit. Although not shown, the control unit of the reader unit may receive data from the signal reader and control the graphical user interface. The reader unit, via the graphical user interface or alternative interface (e.g. buttons), may accept user instructions directly. For example, the user may initiate sensing in this way once the user computer device has provided authorisation and instructions to the control unit.

As shown in FIG. 2, the signal reader may exchange data with the user computer device 2 (labelled APP), e.g. by means suitable APIs. Although not shown, the control unit of the reader unit may receive data from the signal reader and exchange data with the user computer device. As shown, the data may be encoded by an encoder 47 (labelled Encoder) of the reader unit. As shown, the data may be configured to be exchanged via a Bluetooth™ connection 48 (labelled Bluetooth). However, other wireless or wired data connection means may be used instead. For example, WiFi may be used. Accordingly, the reader unit may comprise a data communication unit configured to enable wireless and/or wired communication with the user computer device. Preferably the reader unit is both Bluetooth™ and WiFi enabled.

As shown in FIG. 2, the user computer device may be configured to register the sensor unit against the user as described above. As shown, the user computer device may be configured to identify the type of sensor unit, as described above.

The control unit may be configured to control the reader unit to provide a voltage to the sensor unit. The instructions to the control unit for controlling the reader unit may include a level of the voltage. The control unit may be configured to control the amplitude of the voltage and/or the frequency of the voltage and/or a waveform of the voltage to the sensor unit. Signal generating components of the reader unit may be configured to generate signals with variable voltages and/or variable frequencies and/or variable waveforms. The level of the voltage may be determined based on the information about the identity of the sensor unit. For example, this may be determined based on the type of the sensor unit. Different types of sensor unit may require different input voltage signals to operate.

The control unit may be configured to control the reader unit to perform signal processing on a signal received from the sensor unit. The instructions to the control unit for controlling the reader unit may include a type of signal processing. The type of signal processing is based on the information about the identity of the sensor unit. For example, this may be determined based on the type of the sensor unit. Different types of sensor unit may require different signal processing.

The signal processing may include a comparison of a signal from the sensor unit with baseline sensor data. The instructions to the control unit for controlling the reader unit may include the baseline sensor data. The baseline sensor data may be based on the information about the identity of the sensor unit. For example, this may be determined based on the type of the sensor unit. Different types of sensor unit may have different associated baseline sensor data.

As described above, the user computer device may be configured to determine the type of sensor unit, based on the information about the identity of the sensor unit, and provide instructions to the control unit based on the type of the sensor unit.

As described above, the reader unit may be operable in a plurality of different modes, each different mode corresponding to a different type of sensor unit.

A described above, the instructions provided by the user computer user device to the control unit may be based on data from the host computer device. The host computer device may be configured to provide to the user computer device data relating to one or more of: baseline sensor data, validity of the sensor unit, authorisation for the user to use the sensor unit, validity of the reader unit, authorisation for the user to use the sensor unit.

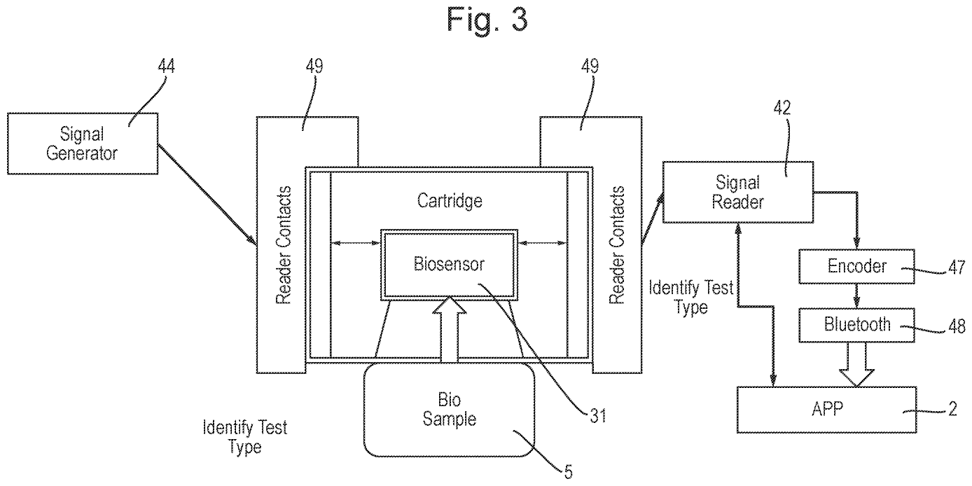

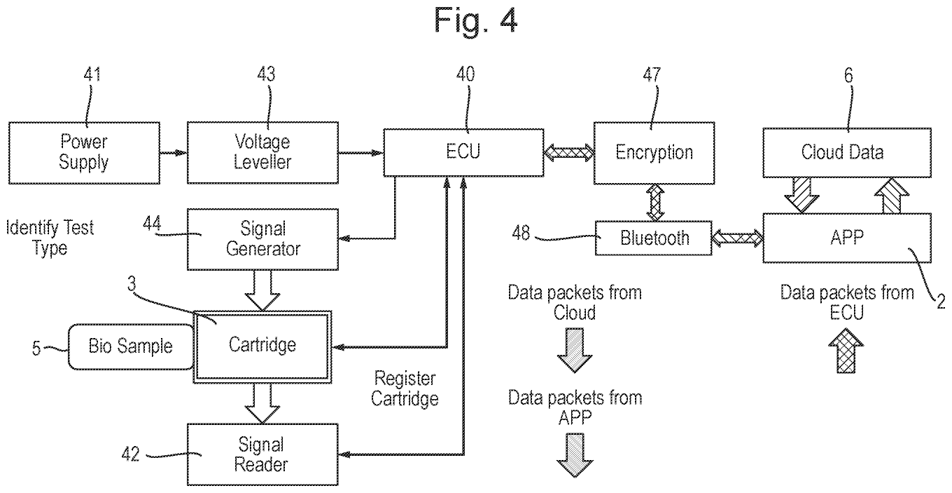

FIG. 4 also shows the example system in further detail. As shown in FIG. 3, data may pass through the control unit 40 (electronic control unit labelled ECU). As shown, in FIG. 3 the user computer device 2 (labelled APP) may exchange data with a host computer device 6 (labelled Cloud Data).

FIG. 3 shows the reader unit and sensor unit in further detail. As shown in FIG. 4, the reader unit and the sensor unit may comprise respective interfaces 49 (labelled Reader Contacts) configured to provide a connection between the reader unit and the sensor unit. As shown, the interfaces may provide electrical connections for providing a voltage signal to the sensor unit and receiving a sensor signal from the sensor unit.

The biosensor may be configured to alter (e.g. attenuate) the voltage signal provided to the sensor unit depending on the amount of the biomarker. Accordingly, the sensor signal is indicative of the amount of biomarker sensed. The biosensor may be a graphene-based biosensor, for example.

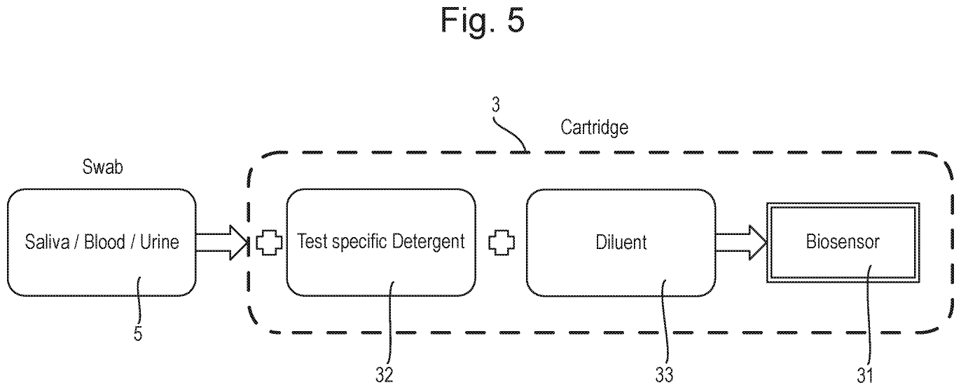

FIG. 5 shows an example sensor unit 3 (labelled Cartridge). As shown, the sensor unit may be configured to receive a biological sample 5 (labelled Swab). As shown, the biological sample may be saliva, blood or urine. Alternatively, the biological sample may be mucous, seminal fluid, vaginal mucous, or any other type of biological sample. These may be human or animal samples. The biological sample may be provided on a swab, provided as a droplet or by any other suitable means to the sensor unit.

As shown, the sensor unit may comprise one or more chemical compositions configured to be mixed with the biological sample. For example, these may include a detergent 32 or a diluent 33, as shown. These may also comprise an aptamer configured to bind to a specific biomarker. These may be specific to the test carried out by the sensor unit. The sensor unit may be configured to mix the biological sample with the one or more chemical compositions. As shown, the mixture may then be provided to the biosensor.

The biological sample may be provided in a sample vessel configured to be received by the sensor unit. The sample vessel may be configured to obtain a sufficient amount of the sample for testing. The sample vessel may comprise the one or more chemical compositions described above for example. The sample vessel may be configured to mix the biological sample with the one or more chemical compositions.

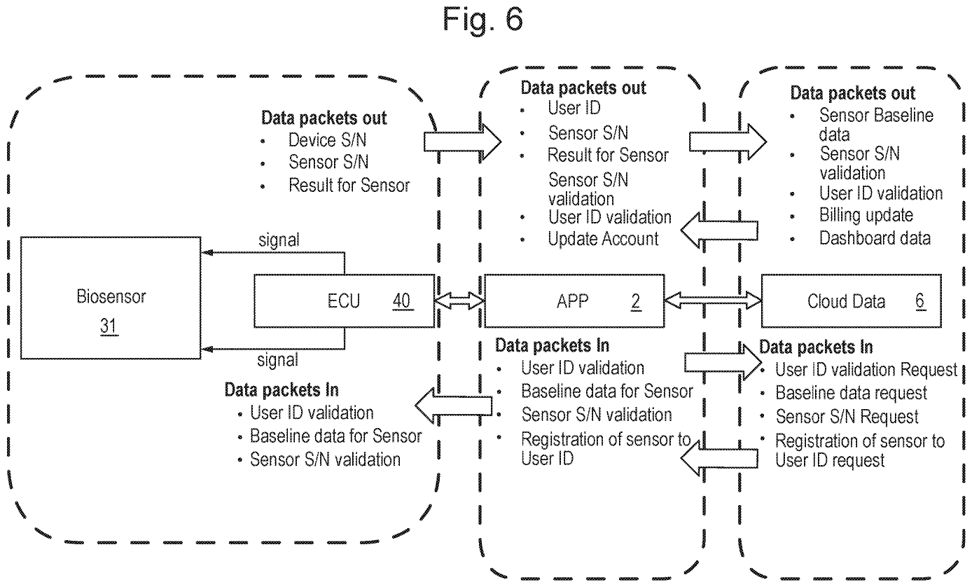

FIG. 6 shows the exchange of data within the diagnostic system. As shown in FIG. 6, the control unit 40 (labelled ECU) or the reader unit may output to the user computer device data relating to the reader unit ID, the sensor unit ID and the sensor signal or result of signal processing. The control unit 40 (labelled ECU) may receive from the user computer device user ID validation, sensor signal baseline data, sensor ID validation, and reader ID validation.

As shown in FIG. 6, the user computer device 4 (labelled APP) may output to the host computer device 6 (labelled Cloud Data) data relating to the user ID, sensor unit ID, reader unit ID, reader unit validation, user account information, user ID. The user computer device may receive from the host computer device data relating to user ID validation, baseline sensor data, sensor unit validation, registration of sensor to use ID.

The system is configured to determine, based on the signal from the sensor unit, whether the biological sample is indicative of a particular state of health or disease. The control unit, the user computer device, or the host computer device may be configured to determine this. Where not determined by the user computer device, the determination is provided to the user computer device from the control unit or the host computer device. The determination may be made based a comparison with one or more baselines, including, for example, population baselines, or user base lines from previous tests.

The above described diagnostics system provides a system whereby users can select a sensor unit corresponding to a specific test, connect the sensor unit to a reader unit compatible with a plurality of different tests, then use the system (i.e. perform the test) via the user computer device. Such a system may be simple and quick to use and may not require specialist knowledge.

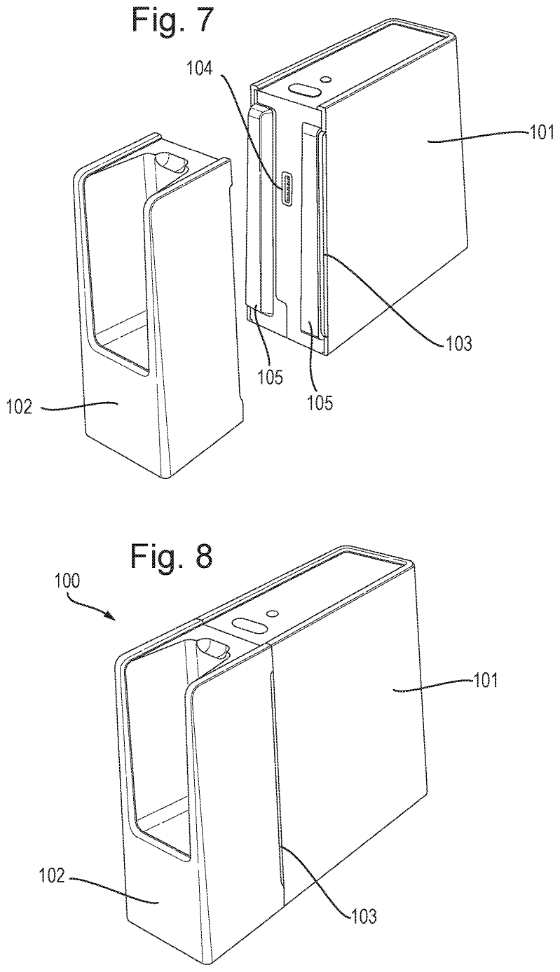

FIGS. 7 and 8 show an example reader unit 100 according to the disclosure. The reader unit may form part for a diagnostics system, such as the example diagnostics systems described above with reference to FIGS. 1 to 6.

As shown the reader unit may comprise a hub unit 101 and an adapter unit 102. The hub unit and the adapter unit are disconnected in FIG. 7 and connected in FIG. 8. As shown, the adapter unit may be configured to disconnectably connect to the hub unit.

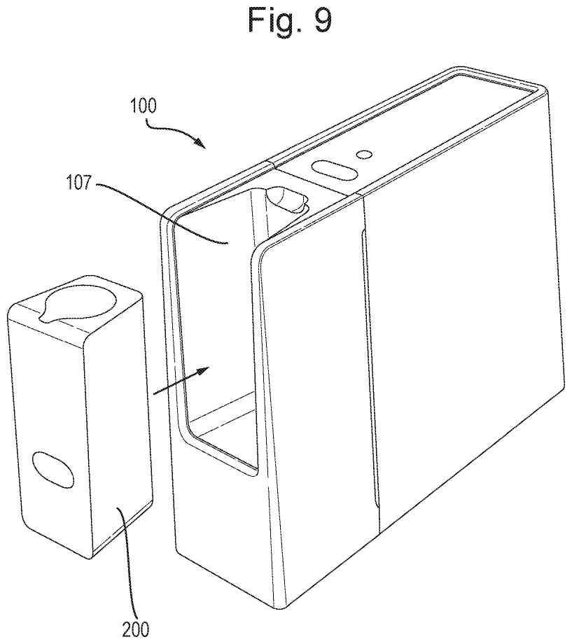

As described above, the reader unit 100 is configured to connect to, and read a signal from, a sensor unit configured to sense a biomarker within a biological sample. As shown in FIG. 9, in the present example, as shown by the arrow, the adapter unit of the reader unit is configured to connect to a sensor unit 200.

As in the example shown, it may be the hub unit that is configured to read the signal from the sensor unit (i.e. when the sensor unit is connected to the adapter unit and the adapter unit is connected to the hub unit). The hub unit may provide a voltage to the sensor unit, as described above in relation to the example systems described with reference to FIGS. 1 to 6. The hub unit may perform signal processing on a signal received from the sensor unit, as described above.

In general, the hub unit may be configured to perform the functions described above as being performed by the reader unit. However, the adapter unit is configured to connect to the sensor unit. Accordingly, the adapter unit provides the interface of the reader unit between the reader unit and the sensor unit, described above.

In the example diagnostics system shown in FIGS. 7 to 9, the adapter unit is configured to be disconnected (and detached) from the hub unit to be replaced by another adapter unit of a different type (not shown). Adapter units of different types may be configured to connect to a respective sensor unit of a different type. Accordingly, the hub unit may be configured to connect to a plurality of adapter units of different types and read a plurality of different signals from different sensor units.

Accordingly, the adapter unit and sensor units are interchangeable, while the hub unit is common for different adapter units. This may simplify manufacturing and reduce the cost of the diagnostics system by further modularising the reader unit.

A kit of parts for assembling a reader unit or diagnostics system may be provided comprising a hub unit and a plurality of adapter units, and optionally a plurality of sensor units.

The adapter unit may comprise a physical interface configured to connect to the sensor unit. The physical interface may be configured to connect to a sensor unit having a corresponding predefined physical form. As shown in FIG. 9, for example, the physical interface may comprise a recess 107 configured to accommodate the sensor unit 200 having a corresponding predefined physical form. Respective different types of adapter unit may be configured to connect with respective different sensor units having respective different predefined physical forms, e.g. to fit different shaped recesses 107 in the adapter unit.

As shown in FIG. 7, the hub unit may comprise a physical interface configured to connect to the plurality of adapter units of different types, e.g. in the form of magnetic protrusions 105 configured to engage with correspondingly shaped magnetic parts of the adapter unit. More generally, the physical interface may comprise one or more of: male and/or female parts configured to connect with corresponding female and/or male parts of the adapter unit, and magnetic parts configured to connect with corresponding magnetic parts of the adapter unit.

As shown in FIG. 7, the hub unit may comprise a signal interface, e.g. in the form of port 104, configured interface communicably with the plurality of adapter units of different types to allow transmission of a signal between the hub unit and the plurality of adapter units. The signal interface may correspond to the data interface and/or power interface as described above in relation to the example systems described with reference to FIGS. 1 to 6. The adapter unit may comprise a first signal interface configured to interface communicably with the hub unit, and a second signal interface configured to interface communicably with the sensor unit, to allow transmission of the signal between the sensor unit and adapter unit.

The adapter unit may be configured to communicate unprocessed signals between the sensor unit and the hub unit. In other words, no signal processing may be performed on the signal form the sensor in the sensor unit between the sensor unit and the hub unit.

Some different types of adapter unit may be configured to interact with the biological sample in the sensor unit. Different types of adapter unit may be configured to interact in respective different ways. For example, the respective different ways may include one or more of: agitation of the biological sample, and heating of the biological sample. Different diagnostic tests may have different requirements. For example, some may require a user to mix a biological sample with a buffer; some may require mixing by the biological sample by the diagnostics system (e.g. by agitation); and some may require heating of the sample to a desired temperature.

As shown in FIGS. 7 and 8, the hub unit may comprise a light source, e.g. LED strip 103 configured to communicate the state of the system to the user. For example, the LED strip may illuminate to communicate that the adapter is connected to the hub, the sensor unit is connected to the adapter, and/or that a signal has been read by the hub from the sensor unit. Different colours and/or patterns of light may be used.

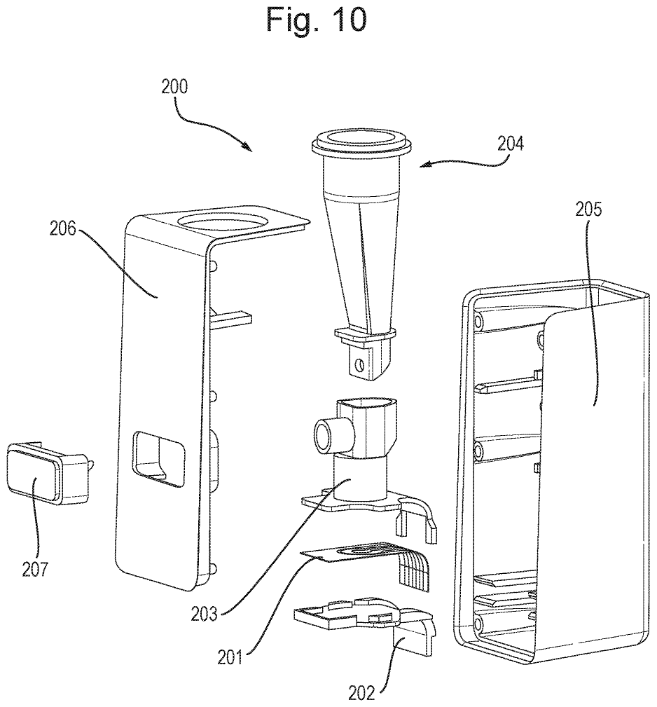

FIG. 10 shows an exploded view of the sensor unit 200 of the example system illustrated by FIGS. 7 to 9. As shown, the sensor unit, comprises a sensor 201. The sensor unit may additionally comprise a sensor mount 202 configured to support the sensor within the sensor unit. The sensor unit may additionally comprise a reservoir 203 and a buffer container 204. As shown, each of the above components 201 to 204 may be housed within a sensor unit housing, e.g. formed from a rear housing part 205 and a front housing part 206.

The buffer container 204 may be configured to contain a chemical composition. The chemical composition may be as described above in relation to the example systems described with reference to FIGS. 1 to 6. The chemical composition may be buffer solution (referred to hereinafter as a buffer). The buffer container is referred to as such for convenience, though it may contain a chemical composition that is not a buffer. The buffer container 204 may be configured to receive the biological sample to form a sample mixture comprising the biological sample and the buffer. The reservoir 203 may be configured to receive the sample mixture from the buffer container and expose the sensor to the sample mixture.

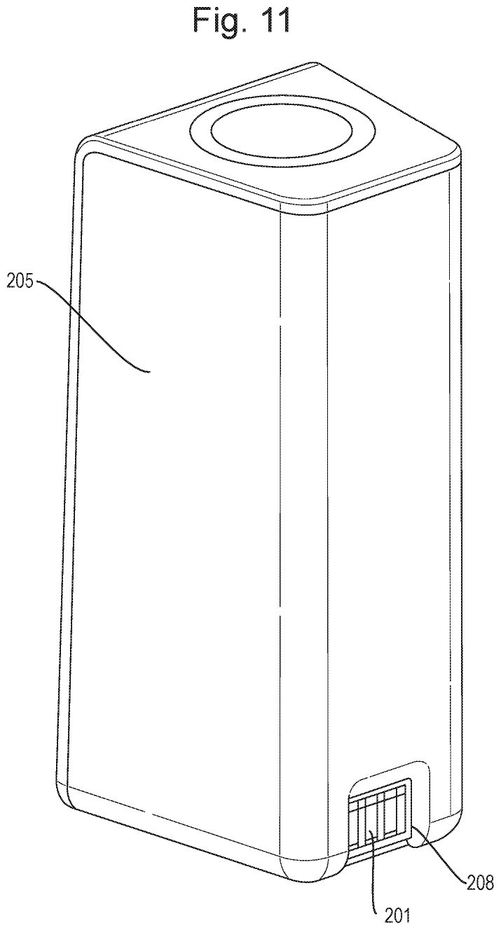

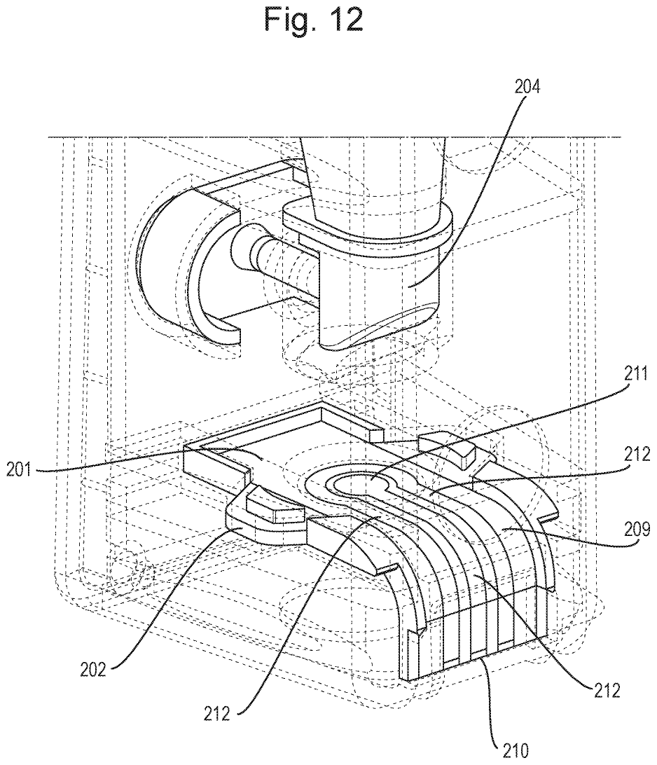

FIG. 11 shows a rear of the sensor unit 200. FIG. 12 shows a partially transparent portion of the rear of the sensor unit 200. As shown in FIG. 12, the sensor 201, may comprise an electrode system 209 integrated with the sensor 201. The electrode system is configured to generate the signal read by the reader unit. As shown in FIGS. 11 and 12, the electrode system may be configured to directly interface communicably with the reader unit through an opening 208 in the sensor housing, e.g. rear housing 205. As shown in FIG. 12, a first portion 210 of the electrode system may be configured to interface with the reader unit 100, e.g. via corresponding electrical contacts of the reader unit (e.g. adapter unit).

As shown in FIG. 12, a second portion 211 of the electrode system may be configured to be exposed to the biological sample. A region of the sensor, at which the second portion of the electrode system is arranged, may be functionalised to bind to the biomarker that the sensor is configured to sense. This functionalised region may comprise a graphene-based material.

As shown in FIG. 12, the electrode system may comprise at least two (e.g. three) electrodes 212 configured to measure the electrical properties of the functionalised region. When the reader unit reads the sensor signal, it may measure said electrical properties.

As shown in FIGS. 10 to 12, the sensor 201 may be bent such that the second portion of the electrode system is arranged substantially in a horizontal plane, in normal use, and the first portion of the electrode system is arranged substantially in a plane at an angle to the horizontal plane, e.g. in a plane perpendicular to the horizontal plane. As shown in FIG. 11, the first portion of the electrode system may be arranged at an outer surface of the sensor unit, substantially parallel to an adjacent portion of the sensor unit housing, e.g. rear housing 205.

In an alternative example system, the sensor may be substantially flat such that the second portion of the electrode system and the first portion of the electrode system are substantially on the same plane. In an example, the first portion may protrude from the sensor unit housing and may be configured to connect to the reader unit via an opening in the reader unit, e.g. the adapter unit. In another example, the first portion may be arranged within the housing and may be configured to connect to the reader unit via a protruding portion of the reader unit configured to extend through the opening in the sensor unit casing.

Features described above in relation to FIGS. 11 to 12 may help to provide a reliable signal interface between the sensor unit and the reader unit.

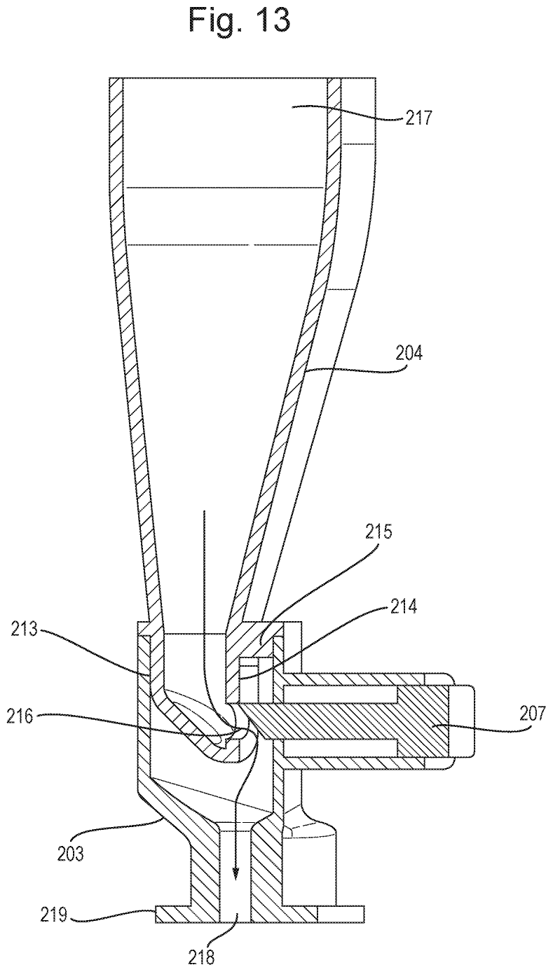

FIG. 13 shows a cross-section through the internal components 201 to 204 of the sensor unit 200. As shown, the buffer container and the reservoir may be integrated within the sensor unit. As shown, the buffer container and the reservoir may be interconnected. For example, as shown, the reservoir may comprise an opening 213 configured to receive a portion 214 of the buffer container. As shown, the opening and the buffer container may be mutually shaped such that the buffer container is retained with the opening. As shown, a further portion of the buffer container, e.g. a collar 215, may substantially seal the opening 213 in the reservoir.

As shown in FIG. 13, the buffer container may comprise an opening 216 through which the sample mixture is configured to be received by the reservoir. As shown, the opening 216 may be arranged at a bottom portion of the buffer container.

The sensor unit 200 may comprise a seal mechanism between the buffer container and the reservoir configured to prevent egress of the buffer or sample mixture from the buffer container to the reservoir through the opening 216. The seal mechanism may be configured to be selectively released to permit egress of the buffer or sample mixture from the buffer container to the reservoir.

As in the example sensor unit shown in FIG. 13, the seal mechanism may comprise a foil or film seal sealing an opening in the buffer container configured to be pierced to release the seal. The sensor unit may comprise a seal release mechanism configured to pierce the seal mechanism. As shown in FIG. 10, a button 207 may be arranged in the sensor housing, e.g. front housing 206. The seal release mechanism may comprise a button actuated protrusion configured to pierce the seal mechanism. This may be actuated by the button 207.

As shown, the buffer container may comprise a further opening 217 configured to receive the biological sample. As shown, the further opening may be arranged at a top portion of the buffer container. The biological sample may be received on a swab inserted into the buffer container, for example. The buffer container may be prefilled with a buffer. The further opening 217 (e.g. prefilled with the buffer), may be sealed by a removable seal, e.g. a film or foil seal. The seal may be peelable, for example.

The buffer container may be configured such that the sample mixture is able to passively flow from the buffer container to the reservoir. For example, the buffer container may be arranged relative to the reservoir such that the sample mixture is configured to passively flow from the buffer container to the reservoir under gravity, in normal use. The buffer container may be arranged substantially above the reservoir, in normal use, as shown.

As shown in FIG. 13, the reservoir comprises an opening 218 through which the sensor unit is exposed and through which the reservoir is configured to expose the sensor to the sample mixture. The reservoir may be configured to substantially form a seal with the sensor to prevent egress of the sample mixture from the opening 218 to a part of the sensor not exposed though the opening. For example, as shown the seal may be formed by a collar 219 surrounding the opening configured to contact the sensor 201.

A part of the reservoir may be configured to direct the mixture revived from the buffer container to the opening 218, e.g. sloped walls leading to the opening 218, as shown. As shown, the reservoir may be arranged relative to the sensor, such that the sample mixture is configured to passively flow from the buffer container to expose the sensor to the sample mixture, e.g. under gravity. The reservoir may be arranged substantially above the sensor, in normal use, as shown.

Features of the sensor unit described with reference to FIG. 13 may help to reliably expose the sensor to the biological sample.

Variations of the above examples are possible within the above teaching without departing from the sprit or scope of the invention.

1. A health monitoring or diagnostics system comprising:
a reader unit configured to connect to, and read a signal from, a sensor unit configured to sense a biomarker within a biological sample, the reader unit comprising a control unit configured to control the reader unit;
a user computer device configured to be operated by a user of the diagnostic system and configured to exchange data with and provide instructions to the control unit for controlling the reader unit,
wherein the control unit is configured to communicate information about an identity of the sensor unit to the user computer device and the user computer device is configured to provide instructions to the control unit for controlling the reader unit based on the identity of the sensor unit.

2. The health monitoring or diagnostics system of any preceding claim,
wherein the control unit is configured to control the reader unit to provide a voltage to the sensor unit, the instructions to the control unit for controlling the reader unit include a level of the voltage, and the level of the voltage is determined based on the information about the identity of the sensor unit.

3. The health monitoring or diagnostics system of any preceding claim,
wherein the control unit is configured to control the reader unit to perform signal processing on a signal received from the sensor unit, the instructions to the control unit for controlling the reader unit include a type of signal processing, and the type of signal processing is based on the information about the identity of the sensor unit.

4. The health monitoring or diagnostics system of claim 3,
wherein the signal processing includes comparison of a signal from the sensor unit with baseline sensor data, the instructions to the control unit for controlling the reader unit include the baseline sensor data, and the baseline sensor data is based on the information about the identity of the sensor unit.

5. The health monitoring or diagnostics system of any preceding claim,
wherein the user computer device is configured to determine the type of sensor unit, based on the information about the identity of the sensor unit, and provide instructions to the control unit based on the type of the sensor unit.

6. The health monitoring or diagnostics system of any preceding claim,
wherein the user computer device is configured to determine whether the user is authorised to use the sensor unit, based on the information about the identity of the sensor unit, and provide instructions to the control unit based on whether the user is authorised to use the sensor unit.

7. The health monitoring or diagnostics system of any preceding claim,
wherein the user computer device is configured to determine whether the sensor unit is valid, based on the information about the identity of the sensor unit, and provide instructions to the control unit based on whether the sensor unit is valid.

8. The health monitoring or diagnostics system of any preceding claim,
wherein the control unit is configured to communicate information about an identity of the reader unit to the user computer device and the user computer device is configured to provide instructions to the control unit for controlling the reader unit based on the identity of the reader unit.

9. The health monitoring or diagnostics system of claim 8,
wherein the user computer device is configured to determine whether the user is authorised to use the reader unit, based on the information about the identity of the sensor unit, and provide instructions to the control unit based on whether the user is authorised to use the sensor unit.

10. The health monitoring or diagnostics system of claim 8 or 9,
wherein the user computer device is configured to determine whether the reader unit is valid, based on the information about the identity of the sensor unit, and provide instructions to the control unit based on whether the sensor unit is valid.

11. The health monitoring or diagnostics system of any preceding claim, comprising a host computer device configured to exchange data with the user computer device,
wherein the instructions provided by the user computer user device to the control unit are based on data from the host computer device.

12. The health monitoring or diagnostic system of claim 11,
wherein the host computer device is configured to provide to the user computer device data relating to one or more of: baseline sensor data, validity of the sensor unit, authorisation for the user to use the sensor unit, validity of the reader unit, authorisation for the user to use the sensor unit.

13. The health monitoring or diagnostics system of any preceding claim,
wherein the reader unit and the sensor unit comprise respective interfaces configured to provide a connection between the reader unit and the sensor unit, the respective interfaces being configured to detachably connect such that the sensor unit can be interchanged with a different sensor unit.

14. The health monitoring or diagnostics system of any preceding claim,
wherein the user computer device is provided separately from the reader unit and is connected wirelessly thereto.

15. The health monitoring or diagnostic system of any preceding claim,
wherein the reader unit is operable in a plurality of different modes, each different mode corresponding to a different type of sensor unit.

16. The health monitoring or diagnostics system of any preceding claim,
wherein the system is configured to determine, based on the signal from the sensor unit, whether the biological sample is indicative of a particular state of health or disease.

17. The health monitoring or diagnostics system of claim 16,
wherein, the control unit, the user computer device, or the host computer device is configured to determine, based on the signal from the sensor unit, whether the biological sample is indicative of a particular state of health or disease.

18. The health monitoring or diagnostics system of claim 16 or 17,
wherein, the determination is provided to the user computer device from the control unit or the host computer device.

19. A health monitoring or diagnostics method comprising:
connecting a sensor unit configured to sense a biomarker within a biological sample to a reader unit comprising a control unit configured to control the reader unit;
the control unit communicating information about an identity of the sensor unit to a user computer device operated by a user of the diagnostic system, and the user computer device providing instructions to the control unit for controlling the reader unit based on the identity of the sensor unit; and
the reader unit reading a signal from the sensor unit.
